(19) United States
(12) Patent Application Publication
(10) Pub. No.: US 2013/0328374 A1
(43) Pub. Date: Dec. 12, 2013

(54) Security Restraint Chair

(71) Applicant: Clifford Lyle Priest, Alpine, CA (US)

(72) Inventor: Clifford Lyle Priest, Alpine, CA (US)

(21) Appl. No.: 13/492,456

(22) Filed: Jun. 8, 2012

(51) Int. Cl. A61F 5/37; A47C 7/02; A47C 7/24; A47C 7/54

(52) U.S. Cl. 297/411.2

(57) ABSTRACT

This invention relates to a security chair that may be used to confine a suspected violent person, such as a criminal, in a courtroom or other setting, where the person must be safely secured for a not insubstantial period of time.

### FIELD OF THE INVENTION

The present invention relates to a chair designed to restrain a person for security reasons.

### BACKGROUND OF THE INVENTION

There are many settings in which it may be desirable to involuntarily restrain a person in a chair for a non-insubstantial period of time. For example, in the field of criminology, there are various settings, in which the criminal may be prone to violence or simply suspected of potential violence, during which it is necessary to confine the person to a chair for a period of hours or longer. In particular, the present invention was conceived and developed for use in a court room where it may be necessary to restrain a defendant in a witness chair during the taking of testimony from the defendant witness.

There are many reports about the violence of criminals that have been incarcerated and when appearing in court for a hearing or trial seize opportunities to escape or injure court personnel including the judge and his or her staff. These include events where the putative criminal has overcome the personnel assigned to escort the person into and out of the courtroom and caused injury or even death of law enforcement personnel. The most dangerous portion of the hearing or trial is when the witness is on the stand testifying about his or her actions or those of another potential criminal since the witness cannot be closely guarded by court personnel responsible for the safety of the courtroom occupants. There are reported instances where the witness, unrestrained except perhaps with handcuffs, has forcibly risen from the chair in the witness box and attacked court personnel. Common restraint devices such as handcuffs, leg iron chains and similar devices that impede movement of the person when walking or being led, do not secure the person in the witness chair preventing his or her arising from the chair and attacking courtroom personnel. Moreover the common witness chair is not usually sufficiently rigid and formed from high strength materials that would permit the attachment of restraining devices to the chair such that the chair could not easily be broken when the witness attempts to break loose from the chair and restraining devices.

There are of course a wide variety of restraint systems and devices used in various applications by law enforcement officials, and in correctional facilities or medical institutions, as well as in court rooms. These devices may include ordinary handcuffs, waist chains, and leg irons, in a wide variety of styles and materials but intended to function to control a combative person in various circumstances including but not limited to the restraint of a person in a chair.

However most of these common devices are not suitable for confining a person in a witness chair in a courtroom for the reasons mentioned above. Among other reasons, obvious restraints may adversely affect the objectivity of a juror by assuming that the accused is dangerous. It would therefore be desirable to provide a simple, effective restraint that is reasonably innocuous and not suggestive that the witness is violent and/or dangerous. It would also be desirable to provide a chair that is formed from rigid high strength materials to which restraining devices may be attached.

The present invention solves this and many other deficiencies in devices and systems that are employed to restrain a suspiciously dangerous person while assuring security for other persons in the vicinity of the person restrained.

### SUMMARY OF INVENTION

A security restraint chair formed of rigid frame members comprising a seat, a back, a pair of arms mounted on each side of the seat, legs supporting the seat above the floor on which the chair rests and a lap restraint comprising a first member or component fixedly attached to the seat and a second member or component, movably, adjustably, connected to the first component and vertically positioned between the legs of a person seated in the chair, and a third member or component attached to the second member and extending horizontally between the arms of the chair and above the thighs of a person seated in the chair.

A method for involuntarily restraining a person in a chair for security reasons comprising the steps of provisioning a rigid chair having a seat, back, and legs supporting the seat above the floor on which the chair rests, and a lap restraint comprising at least a vertical component and a horizontal component attached to the vertical component, seating a person to be restrained in the chair, positioning the vertical component of the lap restraint between the legs of the person, positioning the horizontal component of the lap restraint above the thighs of the person, adjusting the horizontal member closely adjacent the upper surface of the thighs of the person, and locking the lap restraint in position to thereby restrain movement of the person from the chair.

### BRIEF DESCRIPTIONS OF THE DRAWINGS

FIG. 1 is a perspective view of one embodiment of a chair constructed in accordance with the present invention;

FIG. 2 is a second perspective view of the chair shown in FIG. 1 additionally showing various restraint devices usable in connection with the chair;

FIG. 3 is a side elevation view of the chair embodiment of FIG. 1 showing a lap restraint in a pre-assembled position and a slidable foot rest in a forward position;

FIG. 4 is a front view of the chair embodiment shown in FIG. 1.

### DETAILED DESCRIPTION OF ONE EMBODIMENT OF THE INVENTION

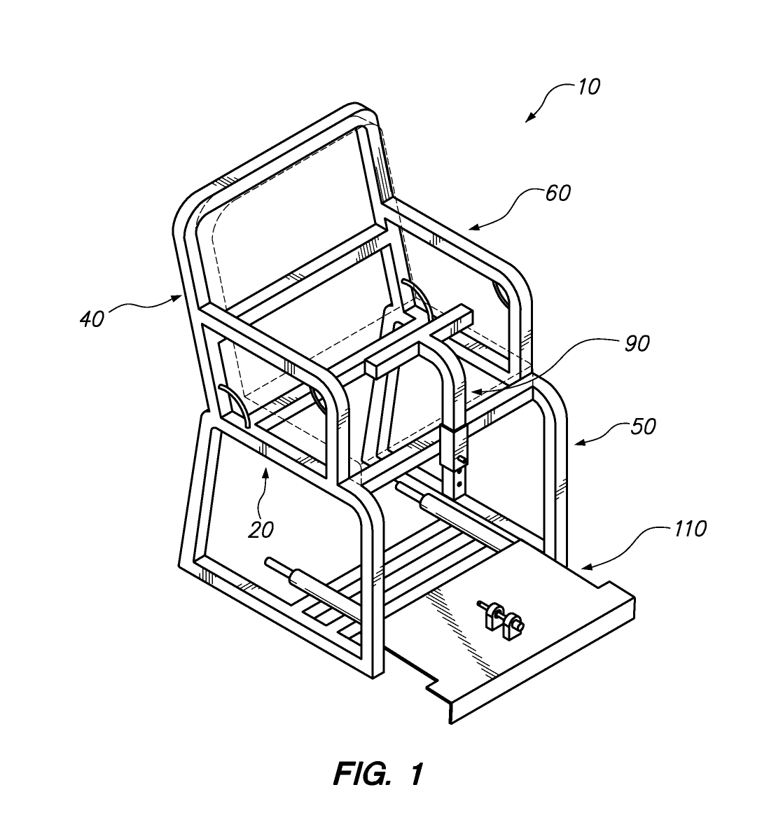

Referring now to FIG. 1, there is shown an embodiment 10 of a chair constructed in accordance with the present invention. The major components or sub-assemblies of the chair embodiment 10 include a seat indicated generally at 20, a back indicated generally at 40, legs indicated generally at 50, arms indicated generally at 60, a lap restraint indicated generally at 90, and a foot rest or support indicated generally at 110.

In embodiment 10, the seat 20, back 40 and legs 50 comprise frame members constructed from a rigid material, such as metal, preferably steel, which may be solid or hollow and may have a square or rectangular cross section as shown. It will be understood by those of ordinary skill in the art that the cross section may be round, elliptical, or other geometric shapes. It will also be understood that the rigid frame members may be formed from materials other than metal, such as plastic or composite materials, that have the requisite strength. The rigid members may have any type of finish, such as paint, anodyne, or plastic coating, or other finishing materials well known in the art. Alternatively to the seat 20 and back 40 comprising frame members, they may be constructed from appropriately sized plates or sheets formed into a seat and back; the plates may be attached to one another or the seat and back may be formed or molded from a single plate or sheet of high strength, rigid material. The legs 50 may also be formed from plate or sheet materials.

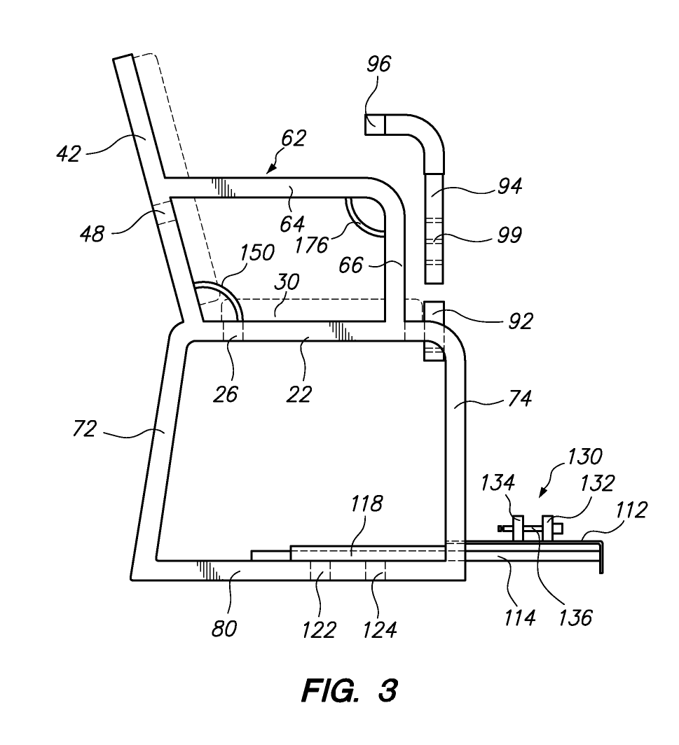

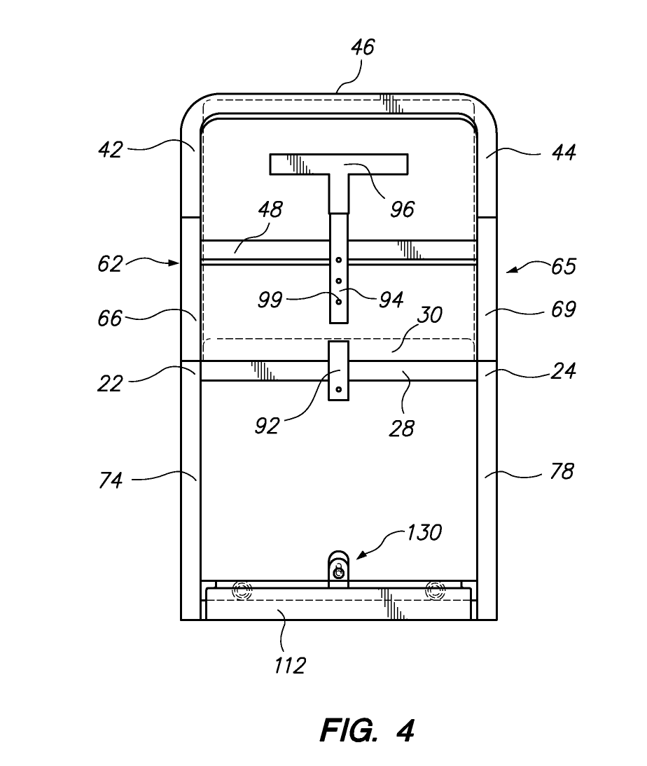

In embodiment 10, as seen best in FIGS. 3 and 4 seat 20 comprises two spaced apart generally horizontally disposed frame members 22, 24 with a rear member 26 and a front member 28. As shown, the seat frame 20 has a generally rectangular configuration and the side members 22, 24 are attached to the rear and front members 26, 28 providing a support structure for a rigid pad shown in dotted lines at 30. The pad 30 may be made of various thicknesses, size, material, and color suitable for the particular application in which the embodiment 10 will be used. As shown, the four seat frame members are rigidly attached, preferably permanently, such as by welding in the event that the frame member material is metal, or may be molded of plastic or fabricated from composite materials so as to provide the proper strength to support persons of size and weight as anticipated for the particular application. Alternatively, in whole or in part, the individual seat frame members 22, 24, 26, and 28 may be separately formed or fabricated and attached to one another semi-permanently, such as by fasteners of various types well known to those having ordinary skill in the art.

The back frame 40 comprises two generally vertical rigid members 42, 44 joined at their upper end by horizontal frame member 46. A stretcher or strength member 48 is horizontally connected between vertical members 42, 44 at a point approximately mid-way from the seat frame 20 to the upper back frame member 46. In embodiment 10, the back frame may comprise frame members that are the same or similar to frame members 22, 24, 26 and 28 that comprise the seat frame 20. The back frame 40 may have a cover, pad, or backing similar to, or part of, the seat pad 30 as described above. The back frame 40 frame members 42, 44, 46 and 48 may be permanently attached similar to the corresponding seat frame members or may be fabricated as individual pieces, and then connected by fastening means as well known in the art.

The chair embodiment 10 additionally includes a pair of side arms 60 including first arm 62 that has a horizontal section 64 and a vertical section 66. Second arm 65 also includes a horizontal section 67 and a vertical section 69. The material, finish, cross section, and other characteristics are the same as or similar to the frame members comprising the seat 20 and the back 40. Arms 62, 65 are positioned on opposite sides of seat 20. Arms 62, 65 may be formed from sheet or plate material that may be individual components attached to seat 20 and/or back 40 or may be integrally formed or fabricated from the same sheet or plate material from which seat 20 and back 40 may be formed.

Chair embodiment 10 additionally includes legs that support the seat 20 above the floor on which the chair rests. The legs 70 includes legs 72, 74 on one side of the chair 10, and 76, 78 on the opposite side. The front and rear legs on each side may be joined at their distal ends by a pair of stretchers 80, 82. In the embodiment shown, the material, finish, cross section and other characteristics of the legs 70 may be identical or similar to the frame members that comprise the seat 20 and the back 40. The legs and stretcher may be integrally formed, for example, if the material is metal, the members may be welded; if plastic, the legs and stretchers may be molded; and if fiber glass or some composite material, they will be fabricated in a manner well known in the art.

Chair embodiment 10 includes a lap restraint 90, that includes a hollow, vertically disposed first restraint component 92 fixedly connected to seat frame front member 28. A second restraint component 94 is vertically disposed and slidably, adjustably inserted into the hollow frame member 92. The lap restraint additionally includes a third restraint component 96 horizontally disposed and connected to an upper portion of the second restraint component 94 and extending horizontally and laterally between the two side arms 62, 65. The vertical position of the second restraint component may be secured or locked in position by a pin (not shown) that is insertable into one of multiple openings 99 in second restraint component 94. If desired, the pin may have a suitable locking mechanism so that only an authorized person can remove the pin after the lap restraint 90 is in position. Those of ordinary skill in the art will appreciate that there are any number of ways of securing a component such as 94 relative to a hollow member 92 after it is inserted in member 92. In the chair embodiment 10 shown, the second and third restraint components are integral and T-shaped, the proper position of the third restraint component 96 above the persons thighs is effected by forming the second and third restraint components 94, 96 so that the second restraint component is bent toward the back 40 of chair 10 thus moving the third restraint component closer to the person's stomach so as to increase the confinement of the lap restraint. Alternatively, the restraint bar 94, 96 may be assembled from separate frame member components and attached by fasteners or the like. Moreover, although the restraint bar 94, 96 may be T-shaped, as shown, the horizontal, laterally extending component 96 may be attached at any upper portion of the vertical second restraint component 94. The particular configuration of the third restraint component is capable of various shapes providing that it performs the function of restraining the person in chair 10 alone or in combination with other restraint devices described below.

The lap restraint 90 may be designed and constructed in various ways with the functional objective of positioning a horizontal component immediately above the seated person's thighs and near the person's stomach so that it is confining but not uncomfortable for the person, and vertical component is positioned between the legs of the person. The first component 92, for example may be a solid shaft and the second component 94 hollow and slidably mounted vertically on the shaft. The permanent or fixed member 92 may be made part of the seat or may be an opening in the seat in which a vertical component may be slidably mounted. The vertical second component may be hingedly attached to the seat and raised into the vertical position after the person is seated. The fixedly mounted first component may be attached to the legs of the chair, rather than the seat. The vertical component may comprise two right-angled members, each pivotally attached to the side arms, and movable from a raised position that allows the person to be seated and to a lowered position such that two vertical members are positioned between the legs of the person seated and the horizontal members are immediately above the person's thighs.

It is preferable that the third horizontally oriented component extend laterally near the side arms so that a thin person cannot escape the confinement by squeezing between the end of the third component and the arm. The third component may be straight as shown, but may also be curved in the horizontal plane to conform more closely to the shape of the stomach of the seated person. It may also be vertically curved to conform more closely to the thighs of the person seated. The third component may be horizontally adjustable toward and away from the back of the chair to be positioned more closely to the seated person's stomach. The third component may be padded for added comfort for the person seated.

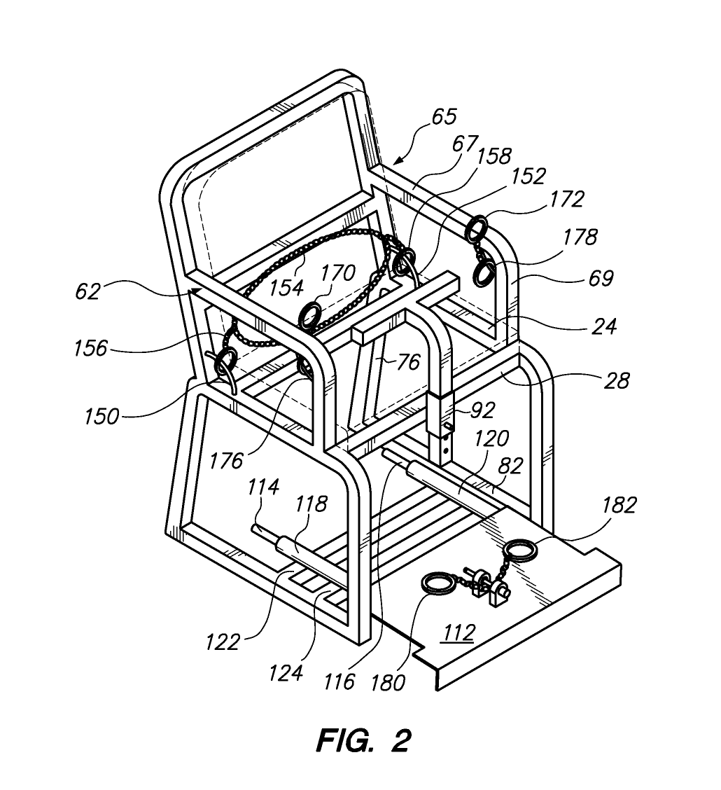

Chair embodiment 10 also includes foot rest or support 110 which comprises a platform 112 mounted on a pair of cylindrical rods 114,116 that are slidably inserted in fixed hollow cylindrical members 118, 120 that are permanently connected to structural members 122, 124. Mounted on the top of platform 112, is a fastening device 130 having an opening through which a leg iron chain may be passed as seen best in FIG. 2. In this particular embodiment, the fastening device comprises a pair of spaced apart ears or lugs 132, 134 through which a pin 136 may be inserted after the leg iron chain is placed between the lugs 132, 134 there by restraining the leg iron chain and preventing movement of the feet of the person seated in chair embodiment 10. In use, it will be understood that the foot rest or support 112 may be slidably adjusted forward and rearward relative to the back of the chair providing a convenient location for the feet of the person seated in the chair while yet effectively restraining the persons feet and legs for security purposes. The position of support 112 may be locked by, for example, providing spaced vertical holes in cylinders 118, 120 and rods 114, 116 and a removable pin.

As shown best in FIG. 2, the chair embodiment 10 may be provided with additional restraining devices which will now be described. Waist restraint members of chair 10 include arcuate members 150, 152 connected at their lower ends to seat frame horizontal members 22, 24, and at their upper ends to back frame vertical members 42, 44 so as to define an opening on each side of the chair, substantially below the side arms 62, 65. As shown, a waist chain 154 that is held in position around the persons waist may then be connected by handcuff halves 156, 158 in a manner well known in the art. As felt necessary, still additional restraint devices may be provided comprising a pair of ordinary handcuffs 170, 172 attached around the wrists of the person seated in the chair and as seen best in FIG. 3, each side arm 62, 65 includes arcuate members 176, 178 similar to members 150, 152, that are attached at one end to vertical sections 66, 69 of the side arms 62, 65 and attached at the other end to side arm horizontal sections 64, 67. Accordingly, the person seated in chair embodiment 10 is prevented from arm movement relative to the chair side arms 62, 65. Those of skill in the art will readily appreciate that the waist and arm restraint members 150, 152, 176, 178 are merely exemplary and that the waist restraint, for example, could be secured to the chair by fastening one of the handcuff halves around one of the seat or back frame members. Alternatively, the seat or back frame members could have openings though which the manacle may be attached. It will be apparent that similar restraint members may be provided on side arms 62, 65 for the wrist handcuff restraints.

In use, the method of involuntarily confining a person in chair embodiment 10 comprises the steps of disassembling the lap restraint 90 so that restraint components 94, 96 are removed from the hollow member 92 as shown best in FIGS. 3 and 4. When the person to be restrained is seated in chair embodiment 10, the lap restraint 90 is positioned between the legs of the person, and the lower end of vertical second component 94 inserted into hollow first component 92, adjusting the height of the lap restraint such that the third horizontal component 96 is in contact with or only slightly above the upper surface of the thighs of the person seated. The pin 99 is then inserted through the opening in the first restraint component 94 and thus locked in position. If additional restraint is required, the person seated may be provided with a waist restraint 54; the person is then restrained from rising from the chair and, depending upon the position of the restraint bar 96 from the stomach of the person, the waist restraint 154 may prevent the person from moving forward in the chair alone or in combination with the restraint bar 96. If still additional restraint is required, then handcuffs 172, 174, attached to the side arms 62, 65 may be attached. Finally, as indicated above, the person may be provided with leg irons 180, 182 and the leg iron chain may be passed through the fastening device 130 restraining movement of the person's feet while allowing the person to move his or her feet forward and rearward through the sliding platform 112. If further confinement is desired, the platform 112 may be locked in position relative to the chair legs by, for example, a pin (not shown) that secures rod 114 at a selected position within cylindrical hollow member 118.

The description of the present invention has been presented for purposes of illustration only, and is not intended to be exhaustive or limited to the embodiments disclosed. Many modifications and variations will be apparent to those having ordinary skill in the art without departing from the scope and spirit of the invention. The example embodiments were chosen and described in order to best explain the principles of the invention and its practical applications, and to enable others of ordinary skill in the art to understand the invention with its various modifications as are suited to the particular use contemplated.

I claim:

1. A chair for involuntarily retaining a person for security reasons comprising:
a seat including a forward edge;
a back;
legs attached to the seat supporting the seat in a raised position from a floor;
a pair of arms attached to the seat and the back on opposite sides of the seat;
said seat forward edge including a vertically disposed hollow member;
a lap restraint member slidably, adjustably mounted in said hollow member, said restraint member projecting above the plane of the seat and toward the back of the chair so as to be positioned between the legs of the seated person and proximate to the person's stomach region; and
restraint devices selected from the group comprising a flexible waist belt, handcuffs, and leg irons attachable to one or more components of the chair.

2. The security chair of claim 1 wherein said seat comprises a rigid, generally rectangular frame including a front member and a padded cover.

3. The chair of claim 2 wherein said back comprises a vertically disposed, rigid generally rectangular frame mounted on the rear edge of the seat.

4. The chair of claim 3 including at least one member attached at one end to the seat frame at a spaced apart location from the back and attached at the other end to the back frame at a spaced apart location from the seat so as to form an opening through which said flexible waist belt may be attached.

5. The chair of claim 1 wherein said vertically disposed hollow member is attached to the seat frame front member.

6. The chair of claim 5 wherein said lap restraint member comprises a T-shaped member bent at an angle comprising a vertical leg adjustably mounted in said hollow member and a horizontally disposed bar extending between said chair arms and above the thighs of the seated person.

7. The security chair of claim 3 wherein each of said arms includes a horizontal section and a vertical section, the horizontal section attached at one end to the back frame, and the vertical section joined at one end to the seat frame, the other ends of the sections joined to one another, and a member attached to the junction of the arm sections to form an opening through which one of the cuffs may be attached around the chair arm and the other cuff around the person's wrist.

8. The security chair of claim 1 wherein said legs are attached by horizontally disposed stretchers parallel to said chair arms.

9. The security chair of claim 8 additionally comprising a foot support mounted between said legs and including a fastening device having an opening though which a leg iron chain may be passed.

10. The security chair of claim 9 wherein said foot support is slidably, adjustably mounted parallel to said arms.

11. The security chair of claim 10 wherein said leg support may be locked in position relative to said chair.

12. The security chair of claim 6 wherein said lap restraint member may be locked in a vertical position so that said horizontally disposed bar is above and closely adjacent the thighs of the seated person.

13. An involuntary restraint chair formed of rigid frame members comprising a seat, a back, a pair of arms mounted on each side of said seat frame, legs supporting the seat above the floor on which the chair rests, and a lap restraint comprising a first component fixedly attached to said seat, a second component, movably, adjustably, connected to said first component and positioned between the legs of a person seated in said chair, and a third component attached to said second component and extending horizontally between said arms and immediately above the thighs of a person seated in the chair.

14. The chair of claim 13 wherein said lap restraint second component may be locked relative to said seat.

15. The chair of claim 13 additionally including at least one wrist restraint attached to the person seated and to the arm of the chair.

16. The chair of claim 13 additionally including means for selectively confining a waist restraint to the back frame.

17. The chair of claim 13 additionally including a leg restraint that is attached to the legs of said chair and passes around one or more legs of the person seated in the chair.

18. The chair of claim 13 additionally including a seat pad and back pad mounted, respectively, on said seat frame and said back frame

19. A method for involuntarily restraining a person in a chair for security reasons comprising the steps of:
provisioning a rigid chair having a seat, back, and legs supporting the seat above the floor on which the chair rests, and a lap restraint attached to the chair, the lap restraint comprising at least a vertical component and a horizontal component attached to the vertical component.
seating a person to be restrained in the chair;
positioning the vertical component of the lap restraint between the legs of the person;
positioning the horizontal component of the lap restraint above the thighs of the person;
adjusting the height of the horizontal component closely adjacent to the upper surface of the thighs of the person; and
locking the lap restraint in position thereby restraining movement of the person from the chair.

20. The method of claim 19 additionally comprising the steps of; restraining the waist of the person; restraining the arms of the person; and/or restraining the feet of the person.
